(19) United States
(12) Patent Application Publication
(10) Pub. No.: US 2020/0188109 A1
(43) Pub. Date: Jun. 18, 2020

(54) COAPTATION ZONE CURTAIN

(71) Applicant: Medtronic Vascular, Inc., Santa Rose, CA (US)

(72) Inventors: Fatemeh Fatemi Far, Santa Rosa, CA (US); Karan Punga, San Rafael, CA (US); Caitlin Dorff, Santa Rosa, CA (US); Emily Grimm, Petaluma, CA (US); Olivia Metcalf, Santa Rosa, CA (US); Matthew E. Genovese, Windsor, CA (US); William Berthiaume, Santa Rosa, CA (US)

(21) Appl. No.: 16/710,867

(22) Filed: Dec. 11, 2019

Related U.S. Application Data
(60) Provisional application No. 62/779,284, filed on Dec. 13, 2018.

(51) Int. Cl. A61F 2/24

(52) U.S. Cl. CPC A61F 2/2445; A61F 2/2466

(57) ABSTRACT

The disclosure describes a medical device comprising a frame configured to substantially conform to a curvature of a coaptation zone of at least two valve leaflets of a cardiac or vascular valve, and a curtain configured to be attached to the frame and extend into the coaptation zone to support coaptation of the at least two valve leaflets.

This application claims the benefit of U.S. Provisional Patent Application Ser. No. 62/779,284, filed on Dec. 13, 2018, the entire content of which is incorporated by reference herein.

### TECHNICAL FIELD

This disclosure relates to heart valve repair, such as mitral valve repair.

### BACKGROUND

Some patient conditions can produce valvular insufficiency or regurgitation.

Valvular insufficiency or regurgitation occurs when a valve in a heart of a subject does not close completely, allowing blood to flow backwards (e.g., from the left ventricle to the left atrium), which may adversely impact the functionality of the heart.

The mitral valve includes two leaflets (anterior and posterior) attached to an annulus (e.g., a fibrous ring). In a healthy heart, the mitral valve leaflets close, or coapt, during contraction of the left ventricle and prevent blood from flowing back into the left atrium. Mitral valve regurgitation is a condition in which the leaflets of a mitral valve of a subject do not coapt properly and, as a result, blood regurgitates back into the left atrium from the left ventricle. The regurgitation of blood back into the left atrium may result in a reduced ejection volume from the left ventricle, causing the heart of the subject to work relatively hard to supply the desirable volume of blood to the body. Mitral regurgitation may occur because of different patient conditions. For example, secondary mitral regurgitation, also referred to as functional mitral regurgitation, may occur when a left ventricle dilates and causes dilation of the mitral annulus of a subject.

### SUMMARY

In some aspects, this disclosure describes example coaptation zone curtains, systems, and techniques for repairing a heart valve, such as, but not limited to, a mitral valve. The coaptation zone curtains, systems, and techniques support coaptation between valve leaflets and may help reduce valvular insufficiency or regurgitation.

In some examples, the disclosure describes a medical device that includes a frame configured to substantially conform to a curvature of a coaptation zone of at least two valve leaflets of a cardiac or vascular valve; and a curtain configured to be attached to the frame and extend into the coaptation zone to support coaptation of the at least two valve leaflets.

In some examples, the disclosure describes a medical device system that includes a delivery device configured to access vasculature of a patient and a medical device. The medical device includes a frame configured to substantially conform to a curvature of a coaptation zone of at least two valve leaflets of a cardiac or vascular valve and a curtain configured to be attached to the frame and extend into the coaptation zone to support coaptation of the at least two valve leaflets. The delivery device is configured to deliver the coaptation zone curtain to the coaptation zone of the cardiac or vascular valve so that the curtain extends between the at least two valve leaflets.

In some examples, the disclosure describes a method that includes advancing a delivery device through vasculature of a patient to a vascular or cardiac treatment site. The delivery device includes a lumen housing a coaptation zone curtain. The coaptation zone curtain includes a frame configured to substantially conform to a curvature of a coaptation zone of at least two valve leaflets of a cardiac or vascular valve and a curtain configured to be attached to the frame and extend into the coaptation zone to support coaptation of the at least two valve leaflets. The method also includes releasing the coaptation zone curtain from the lumen to the coaptation zone of the cardiac or vascular valve so that the curtain extends between the at least two valve leaflets.

The details of one or more examples are set forth in the accompanying drawings and the description below. Other features, objects, and advantages of examples according to this disclosure will be apparent from the description and drawings, and from the claims.

### BRIEF DESCRIPTION OF THE DRAWINGS

FIGS. 1A and 1B are schematic cross-sectional views of an example human heart.

FIG. 2 is a schematic cross-sectional view of a coaptation zone of an example human heart.

FIG. 3 is an image of an example coaptation zone curtain including a frame and a curtain.

FIGS. 4A and 4B are two images of an example frame for a coaptation zone curtain.

FIGS. 5A-5C are schematic views of example frames for coaptation zone curtains.

FIG. 6 is an image of an example coaptation zone curtain, including a frame and a curtain.

FIGS. 7A-7E are schematic views of example anchors for a coaptation zone curtain.

FIGS. 8A and 8B are schematic views of an example delivery device and an example coaptation zone curtain.

FIG. 9 is a schematic view of an example coaptation zone curtain including a three-dimensional frame and curtain.

FIG. 10 is a schematic view of an example coaptation zone curtain including a three-dimensional frame and curtain.

FIG. 11 is a flow diagram illustrating an example technique for implanting an coaptation zone curtain.

FIG. 12 is an image of an example wire frame for a coaptation zone curtain.

FIG. 13 is an image of an example wire frame for a coaptation zone curtain.

FIG. 14 is an image of an example wire frame for a coaptation zone curtain engaged with a model of a mitral valve.

### DETAILED DESCRIPTION

This disclosure describes devices, systems, and techniques for repairing a heart valve, such as, but not limited to, a mitral valve. The coaptation zone curtains, systems, and techniques support coaptation of valve leaflets of the heart valve, e.g., help provide better (e.g., more effective) coaptation of the valve leaflets, which may help and may help reduce valvular insufficiency or regurgitation for a particular patient. For example, the coaptation zone curtains may be configured to increase a length over which the valve leaflets coapt, which may provide more effective coaptation of the valve leaflets. Coaptation zone curtains may include a frame and a curtain attached to the frame. In some examples, a coaptation zone curtain also may include at least one of an alignment marker, a barb configured to engage tissue, or at least one anchor. The frame may be configured to engage with tissue of an annulus of the heart valve, a trigone of the heart valve, and/or a valve leaflet to position the curtain within the coaptation zone, and may be formed from a wire, cut sheet (e.g., a laser-cut sheet of material), or the like. The curtain may be configured to extend into the coaptation zone, increase a coaptation length between the valve leaflets, and provide support to the coaptation zone.

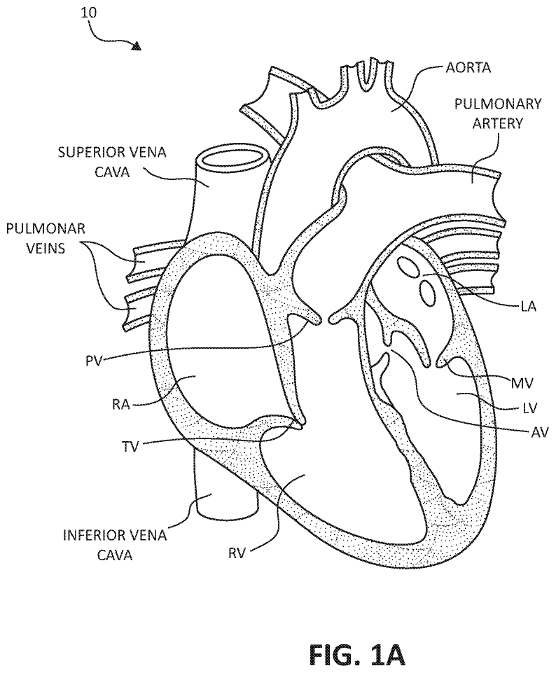

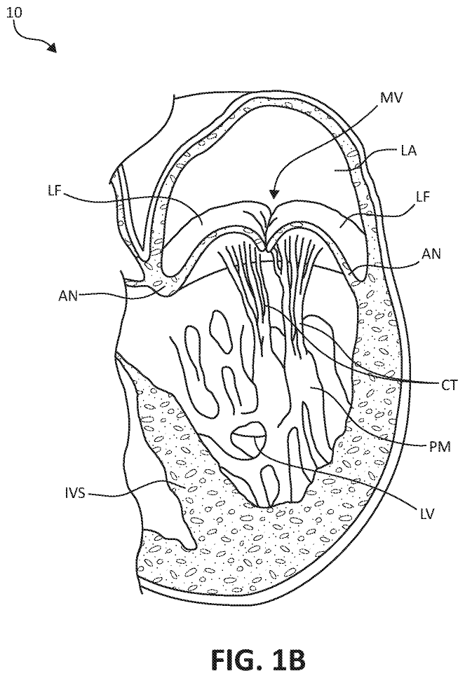

FIGS. 1A and 1B are schematic cross-sectional views of an example human heart 10. The human heart 10 is a four chambered, muscular organ that provides blood circulation through the body during a cardiac cycle. The four main chambers include the right atrium (RA) and right ventricle (RV) which supplies the pulmonary circulation, and the left atrium (LA) and left ventricle (LV) which supplies oxygenated blood received from the lungs to the remaining body. To ensure that blood flows in one direction through the heart, atrioventricular valves (tricuspid valve (TV) and mitral valves (MV)) are present between the junctions of the atrium and the ventricles, and semi-lunar valves (pulmonary valve (PV) and aortic valve (AV)) govern the exits of the ventricles leading to the lungs and the rest of the body. These valves contain leaflets (LF) or cusps that open and shut in response to blood pressure changes caused by the contraction and relaxation of the heart chambers. FIG. 1B is a schematic sectional illustration of a left ventricle LV of heart 10 showing anatomical structures and a native mitral valve MV.

The left atrium LA receives oxygenated blood from the lungs via the pulmonary veins and pumps the oxygenated blood through the mitral valve MV and into the left ventricle LV during ventricular diastole. The left ventricle LV contracts during systole and blood flows outwardly through the aortic valve AV, into the aorta and to the remainder of the body. In a healthy heart, the leaflets LF of the native mitral valve MV meet evenly at the free edges or “coapt” to close and prevent back flow of blood into the left atrium LA during contraction of the left ventricle LV. The tissue of the leaflets LF attach to the surrounding heart structure via a dense fibrous ring of connective tissue called an annulus AN. The flexible tissue of the leaflets LF of the native mitral valve MV are connected to papillary muscles PM, which extend upwardly from the lower wall of the left ventricle LV and the interventricular septum IVS, via branching tendons called chordae tendinae CT.

Mitral valve regurgitation is a condition in which the leaflets of a mitral valve of a subject do not coapt properly and, as a result, blood regurgitates back into the left atrium LA from the left ventricle LV. The regurgitation of blood back into the left atrium LA may result in a reduced ejection volume from the left ventricle LV, causing the heart of the subject to work relatively hard to supply the desirable volume of blood to the body. Mitral regurgitation may occur because of one or more patient conditions. For example, secondary mitral regurgitation, also referred to as functional mitral regurgitation, may occur when the left ventricle LV dilates and causes dilation of the mitral annulus of a subject. The leaflets LF of the valves may move apart as a result of the dilation of the left ventricle LV, which may adversely impact the ability of the leaflets to properly coapt.

In addition to or instead of being caused by dilation of the left ventricle LV, mitral valve regurgitation (or other valve regurgitation) may be caused by calcified plaque buildup in heart 10. For example, the leaflets LF of the valves (e.g., aortic valve AV or mitral valve MV) may harden and may not sufficiently coapt or meet, such that regurgitation may occur where the valve does not close completely, allowing blood to flow backwards (e.g., from the left ventricle LV to the left atrium LA). The left side of heart 10 (e.g., mitral valve MV and aortic valve AV) can be more likely to become calcified because of the higher pressures generated.

In addition to or instead of being caused by dilation of the left ventricle LV or calcified plaque build-up, mitral valve regurgitation (or other valve regurgitation) may be caused leaflet perforation, vegetation, tear, chordae tendineae rupture and elongation, papillary muscles rupture and elongation, and ventricle infarction.

The medical devices, systems, and techniques described herein may be used to repair a valve of heart 10 via a minimally invasive medical procedure, e.g., via a transcatheter medical procedure that is less invasive than open heart surgery. While open heart surgeries, such as annuloplasty performed via open heart surgery, may have positive outcomes, a more minimally invasive medical procedure may also have positive outcomes while also being associated with a shorter recovery time for some patients compared to open heart surgery.

Although example devices, systems, and techniques are primarily described herein with reference to the mitral valve MV, in other examples, the example devices, systems, and techniques may be used to repair other valves in heart 10.

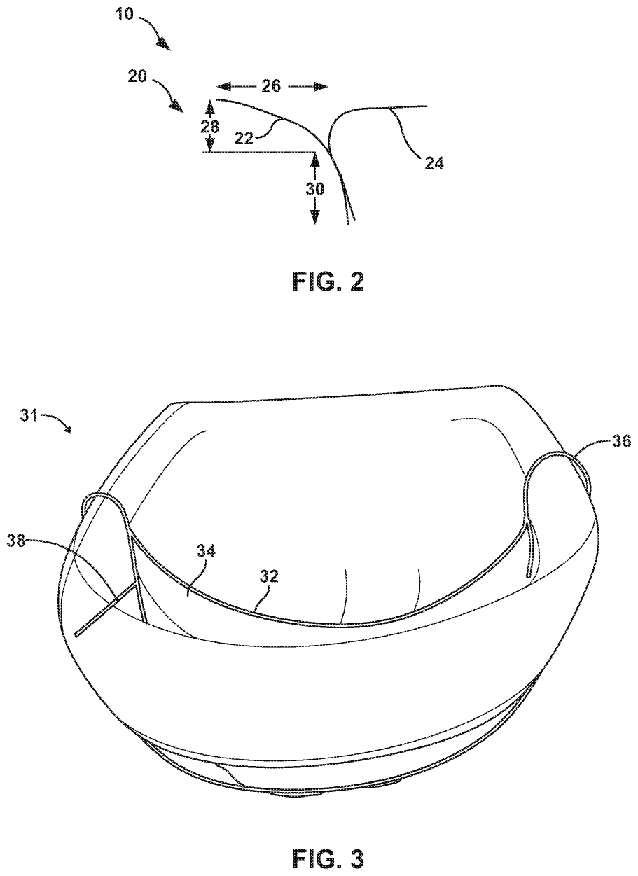

FIG. 2 is a schematic cross-sectional view of a coaptation zone 20 of an example human heart 10. FIG. 2 illustrates an anterior leaflet 22 and posterior leaflet 24 of a mitral valve. Coaptation zone 20 defines a coaptation distance 26, a coaptation depth 28, and a coaptation length 30. Coaptation distance 26 can be defined, for example, as a shortest linear distance between one end of a valve annulus AN and the location where the valve leaflet overlaps with another valve leaflet LF. In some examples, the coaptation depth 28 is measured in a medial-lateral direction. The coaptation depth 28 can be defined, for example, as a shortest linear distance, measured in a direction perpendicular to the direction in which the coaptation distance 26 is measured, between one end of a valve annulus AN and the location where the valve leaflet overlaps with another valve leaflet LF. In some examples, the coaptation depth 28 is measured in a superior-inferior direction. The coaptation length 30 can be defined, for example, as a linear distance that the valve leaflets LF overlap when coapted. In some examples, the coaptation length 30 is measured in a direction perpendicular to the direction in which the coaptation distance 26 is measured, and is measured in a superior-inferior direction.

The coaptation zone curtains of the current disclosure may be configured to provide mechanical support to coaptation zone 20 and increase coaptation length 30 between anterior leaflet 22 and posterior leaflet 24. In this way, the coaptation zone curtains of the current disclosure may help increase heart valve leaflet coaptation, which may reduce or even eliminate mitral regurgitation. In some examples, a coaptation zone curtain includes a curtain that is configured to extend between the at least two valve leaflets of a valve in order to increase the coaptation length 30 between the leaflets.

FIG. 3 is an image of an example coaptation zone curtain 31 including a frame 32 and a curtain 34. Frame 32 is configured to support curtain 34 and position curtain 34 within a coaptation zone (e.g., coaptation zone 20 of FIG. 2). For example, frame 32 may have a curvature, length, and width selected to substantially conform to curvature of at least a portion of coaptation zone 20.

Frame 32 may include a biocompatible material. In some examples, frame 32 is configured to substantially maintain its shape under forces experienced by frame 32 once frame 32 is deployed in heart 10 (FIGS. 1A and 1B). In some examples, frame 32 may include a biocompatible metal or alloy, such as nitinol, stainless steel, a cobalt-chromium alloy, or the like. In other examples, frame 32 may include a biocompatible polymer, or a biocompatible metal-polymer composite. In some instances, frame 32 may include a biocompatible shape memory alloy. Frame 32 may be formed using any suitable technique, including bending a wire, cutting (e.g., laser-cutting) a sheet, or the like

In some examples, frame 32 may have a pre-set shape. For example, the pre-set shape may be defined using a heat treatment. The pre-set shape is a shape toward which frame 32 recovers in the absence of an applied force. In some examples, the pre-set shape may substantially conform to curvature of at least a portion of coaptation zone 20. The pre-set shape of frame 32 also may be configured to extend curtain 34 into the coaptation zone between at least two valves (e.g., coaptation zone 20 of FIG. 2). In some examples, frame 32 may define a substantially two-dimensional (e.g., planar) shape. In other examples, frame 32 may define a three-dimensional shape. In some examples in which frame 32 defines a three-dimensional shape, a length of frame 32, a depth of frame 32, a width of frame 32, a curvature of frame 32, and a placement of curtain 34 are selected so that curtain 34 substantially fills a gap between the at least two valve leaflets and provides a sealing surface between the at least two valve leaflets.

Curtain 34 is attached to frame 32 using any suitable technique. For example, curtain 34 may be sewn to frame 32. As other examples, curtain may be one or more of adhered, clipped or welded to frame 32. Curtain 34 may be attached to frame 32 at a position such that curtain 34 extends into coaptation zone 20 when frame 32 is positioned contacting tissue of the cardiac or vascular valve. For example, curtain 34 may extend into the portion of coaptation zone 20 indicated by coaptation length 30. Curtain 34 may be configured to provide a surface against which valve leaflets LF (FIGS. 1A and 1B), such as anterior leaflet 22 and posterior leaflet 24 (FIG. 2) may contact or coapt. In this way, curtain 34 may support or improve coaptation of valve leaflets LF (FIGS. 1A and 1B), such as anterior leaflet 22 and posterior leaflet 24 (FIG. 2).

Curtain 34 may include any suitable biocompatible material. In some examples, curtain 34 may include a porous material. In other examples, curtain 34 may include a substantially non-porous material. Curtain 34 may include at least one of pericardial tissue, a biocompatible polymer, or a biocompatible fabric.

In some examples, coaptation zone curtain 31 (e.g., frame 32) may include at least one barb 36. At least one barb 36 may be configured to engage tissue of the cardiac or vascular valve, such as annulus AN (FIGS. 1A and 1B), a trigone, or a leaflet LF. At least one barb 36 may help maintain frame 32 in place once frame 32 is positioned as desired. At least one barb 36 may be integrally formed with frame 32 or may be formed separately and attached to frame 32 (e.g., welded, adhered, mechanically connected, or the like). Coaptation zone curtain 31 may include any number of barbs 36, such as at least one, at least two, two, or the like.

In some examples, coaptation zone curtain 31 (e.g., frame 32) may include at least one alignment marker 38. At least one alignment marker 38 may be configured to facilitate alignment of frame 32 with coaptation zone 20. At least one alignment marker 38 may be integrally formed with frame 32 or may be formed separately and attached to frame 32 (e.g., welded, adhered, mechanically connected, or the like). In some examples, at least one alignment marker 38 may be adjacent at least one barb 36 to help align the at least one barb 38 with a trigone or with annulus AN. Coaptation zone curtain 31 may include any number of alignment markers 38, such as at least one, at least two, two, or the like.

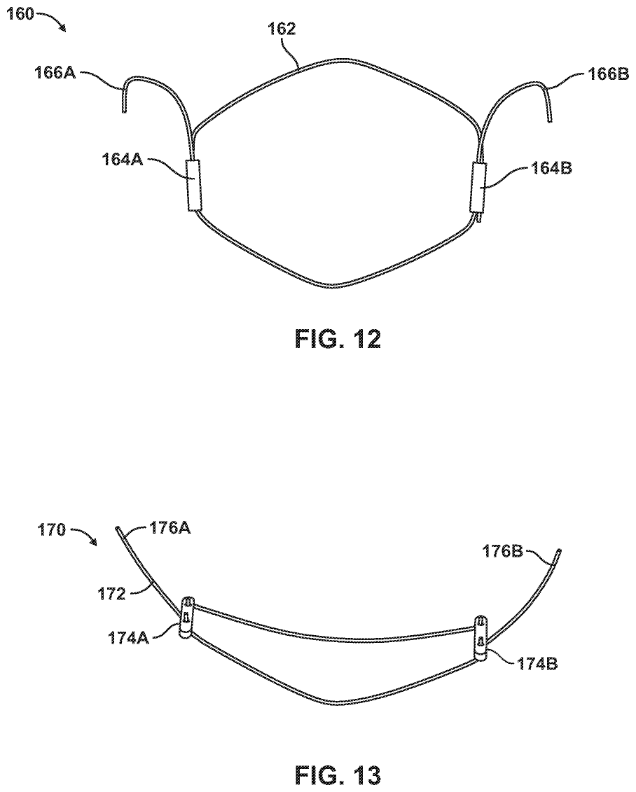

FIGS. 12 and 13 are images of example frames 162 and 172 for example coaptation zone curtains 160 and 170, respectively. Frames 162 and 172 may be similar to or substantially the same as any other frames described herein, including frame 32 of FIG. 3, aside from the differences described herein. Frames 162 and 172 include alignment markers 164A, 164B and 174A, 174B, respectively. Respective alignment markers 164A, 164B and 174A, 174B may be adjacent respective barbs 166A, 166B, 176A, 176B to help align the barbs 166A, 166B, 176A, 176B with a trigone or with annulus AN. Although not shown in FIGS. 12 and 13, coaptation zone curtains 160 and 170 may each include a curtain, e.g., any of the curtains described above.

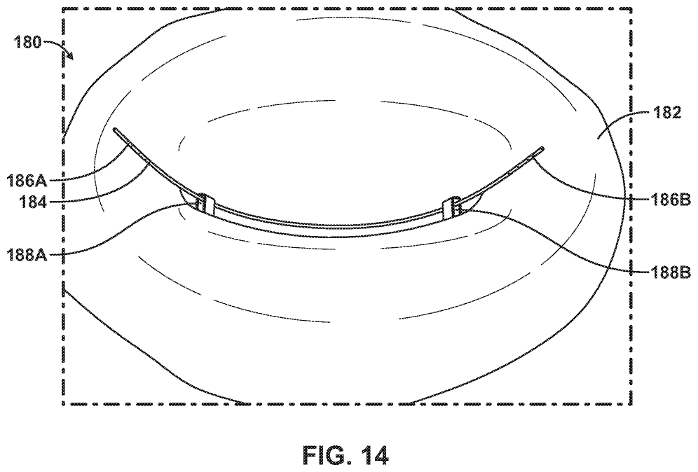

FIG. 14 is an image of an example coaptation zone curtain 180 engaged with a model of a mitral valve 182. Coaptation zone curtain 180 may be similar to or substantially the same as any other coaptation zone curtains described herein, including coaptation zone curtain 30 of FIG. 3, aside from the differences described herein. Coaptation zone curtain 180 includes a frame 184. Frame 184 includes first and second barbs 186A and 186B, and respective alignment markers 188A and 188B adjacent to respective barbs 186A and 186B.

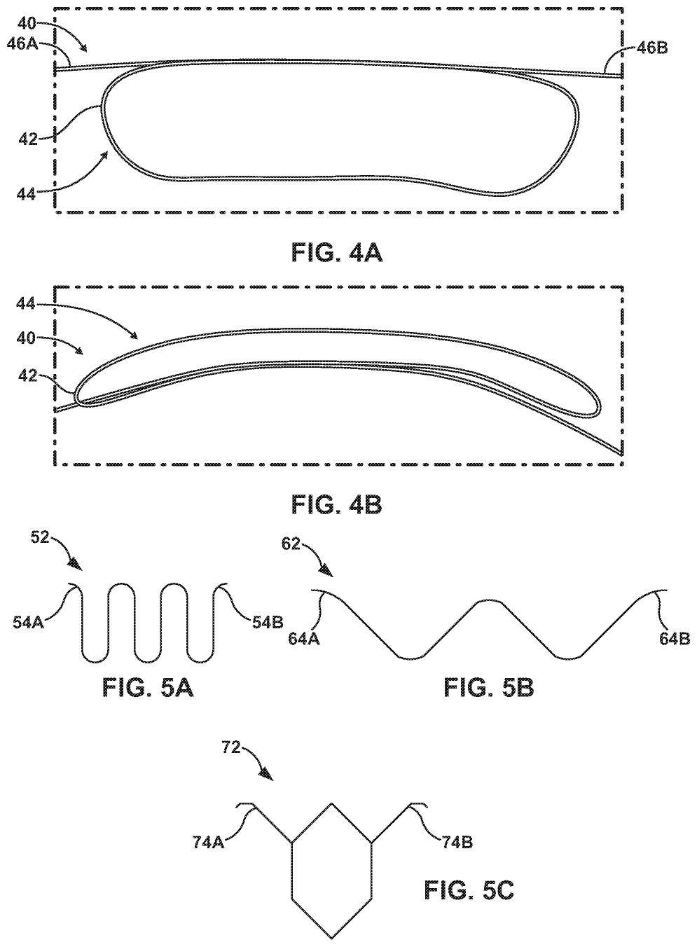

Returning to FIG. 3, frame 32 may have any suitable shape configured to engage with tissue near coaptation zone 20, e.g., to substantially conform to at least a portion of coaptation zone 20. FIGS. 4A and 4B are images of an example frame 42 of an example coaptation zone curtain 40. FIG. 4A is a side view image and FIG. 4B is a perspective view image. As shown in FIGS. 4A and 4B, frame 42 defines a loop 44 with two extensions 46A and 46B. The two extensions 46A and 46B may be configured to engage with tissue near coaptation zone 20, e.g., to substantially conform to at least a portion of coaptation zone 20. Loop 44 may be configured to extend into coaptation zone 20, e.g., to the portion of coaptation zone 20 indicated by coaptation length 30 in FIG. 2. Further, a curtain may be attached to loop 44. Frame 42 is curved as shown in FIG. 4B to better conform to at least a portion of coaptation zone 20, e.g., near coaptation length 30.

In other examples, a frame may have a different shape. FIGS. 5A-5C are schematic views of example frames 52, 62, and 72 for coaptation zone curtains.

Frame 52 shown in FIG. 5A includes an undulating (e.g., serpentine or sinusoidal) shape and barbs 54A and 54B. The undulating shape of frame 52 may support a curtain more completely, e.g., along a length of the curtain, than a more open shape such as loop 44.

Frame 62 shown in FIG. 5B also includes an undulating (e.g., a serpentine or sinusoidal shape) and barbs 64A and 64B. The undulating shape of frame 62 is more widely spaced than the sinusoidal shape of frame 52.

Frame 72 shown in FIG. 5C includes a polygonal shape and barbs 74A and 74B. Although frame 72 has a hexagonal shape, frame 72 may include any other hexagonal shape or other shapes formed from interconnected struts.

Although not shown in FIGS. 5A-5C, frames 52, 62, and 72 may include curvature in the dimension into and out of the page of FIGS. 5A-5C, similar to frame 42 of FIGS. 4A and 4B.

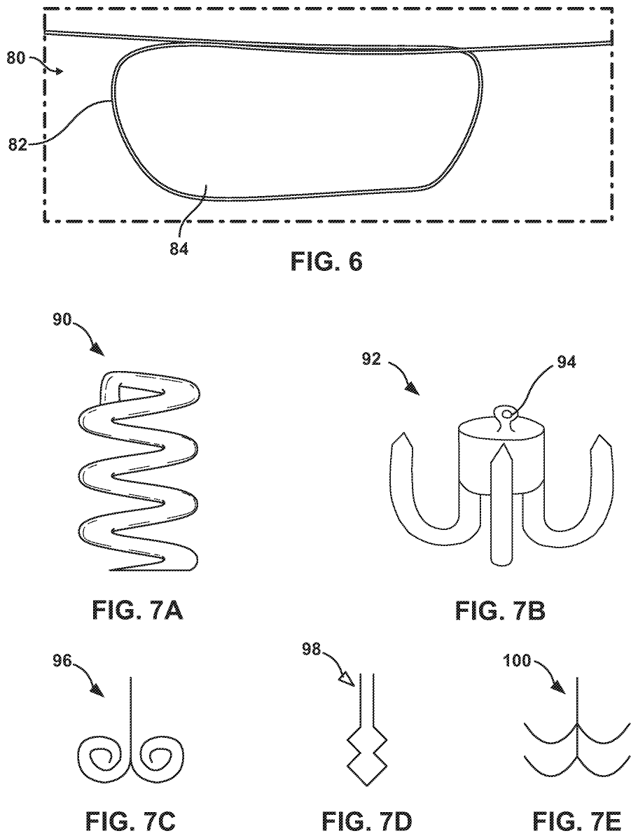

FIG. 6 is an image of an example coaptation zone curtain 80, including a frame 82 and a curtain 84. Frame 82 may be similar to or substantially the same as frame 42 of FIG. 4 in some examples. Curtain 84 is attached to loop 86 of frame 82, e.g., sewn to loop 86 of frame 82, adhered to frame 82, welded to frame 82, or attached using other suitable techniques.

In some examples, a coaptation zone curtain may be anchored to tissue of heart 10 (FIGS. 1A and 1B) using at least one anchor, in addition to or instead of at least one barb. The at least one anchor may include any structures configured to engage the frame to tissue, e.g., tissue of the annulus AN and/or leaflets LF, and retain the frame substantially in place relative to annulus AN and/or leaflets LF. FIGS. 7A-7E are schematic views of example anchors for a coaptation zone curtain.

In some examples, the at least one anchor includes a helix 90, as shown in FIG. 7A, or a double helix that is configured to be advanced into tissue of heart 10, e.g., annulus AN and/or leaflets LF. In other examples, the at least one anchor includes a multi-pronged hook 92, as shown in FIG. 7B. In other examples, the at least one anchor may include a fish bone anchor 96 or 100, as shown in FIGS. 7C and 7E, or a harpoon anchor 98, as shown in FIG. 7D. The anchor may optionally include an attachment feature 94 (FIG. 7B), such as a hook, loop, or the like that is configured to engage the frame to substantially retain the frame relative to the anchor. In this way, the at least one anchor may be configured to substantially retain the frame relative to a location of tissue of heart 10, such as a location of annulus AN and/or leaflets LF.

The at least one anchor may include a biocompatible material that is configured to engage the frame. In some examples, the at least one anchor may include a biocompatible metal or alloy, such as nitinol, stainless steel, a cobalt-chromium alloy, or the like. In some instances, the at least one anchor may include a biocompatible shape memory alloy.

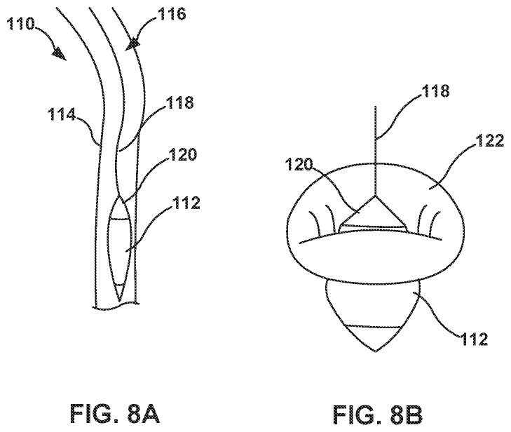

The coaptation zone curtains described herein may be delivered to the coaptation zone using a delivery device. FIGS. 8A and 8B are schematic view of an example delivery device 110 and a coaptation zone curtain 112. In the example of FIGS. 8A and 8B, delivery device 110 includes a catheter 114. Catheter 114 may define an internal lumen 116 that extends from proximate a proximal end of catheter 114 to proximate a distal end of catheter 114 (e.g., may extend from the proximal end to the distal end). Lumen 116 may be configured to house coaptation zone curtain 112 during percutaneous introduction of catheter 114 into vasculature of a patient and advancing of the distal end of catheter 114 to the treatment site, such as the left atrium LA (FIGS. 1A and 1B). In some examples, catheter 114 may be used with a guidewire, a guide catheter, a dilator, or the like, to facilitate introducing catheter 114 into vasculature of a patient and advancing the distal end of catheter 114 to the treatment site. In some examples, catheter 114 includes a steerable shaft and/or distal tip to allow a clinician to control positioning of the distal tip relative to anatomical structures, such as heart 10. In some implementations, catheter 114 may access left atrium LA trans-septally, trans-aortically, or trans-apically.

Catheter 114 is also configured to deploy coaptation zone curtain 112 in position proximate coaptation zone 20 (FIG. 2) of mitral valve 122 and engage the frame of coaptation zone curtain 112 with tissue of heart 10. In some examples, to facilitate positioning of catheter 114, coaptation zone curtain 112, or both, within the treatment site, a distal portion of catheter 114 may include at least one radiographic marker configured to be visualized using a radiographic technique.

In some examples, delivery device 110 additionally may include a balloon catheter 118 that includes an expandable balloon 120. Coaptation zone curtain 112 may be folded, wrapped, or crimped around expandable balloon 120 when expandable balloon 120 is in a folded or retracted state. Coaptation zone curtain 112 and expandable balloon catheter 118 may be advanced through lumen 116 of catheter 114 and out a distal exit of lumen 116. Once coaptation zone curtain 112 and expandable balloon 120 are released from lumen 116 and adjacent the desired position for coaptation zone curtain 112, expandable balloon 120 may be expanded as shown in FIG. 8B to deploy coaptation zone curtain 112.

In other examples, the frame (e.g., frame 32 of FIG. 3 or other frames described herein) of coaptation zone curtain 112 is self-expandable and may be held in a lower profile delivery configuration when within lumen 116 of catheter 114 and may be configured to expand into a deployed configuration once released from lumen 116. In some of these examples, coaptation zone curtain 112 may be deployed without the aid of expandable balloon 120. In other examples, however, expandable balloon may also be used to deploy a coaptation zone curtain 112 including a self-expandable frame.

Delivery device 110 also may be used by a clinician to engage at least one barb FIG. 7 (FIG. 3) and/or at least one anchor (FIGS. 7A and 7B) with the frame of coaptation zone curtain 112 and tissue of heart 10. In other examples, a separate anchor delivery device may be used by the clinician to engage at least one anchor (FIGS. 7A and 7B) with the frame of coaptation zone curtain 112 and tissue of heart 10. For example, the clinician may withdraw balloon catheter 118 through lumen 116 and advance the anchor delivery device through lumen 116 to the treatment site.

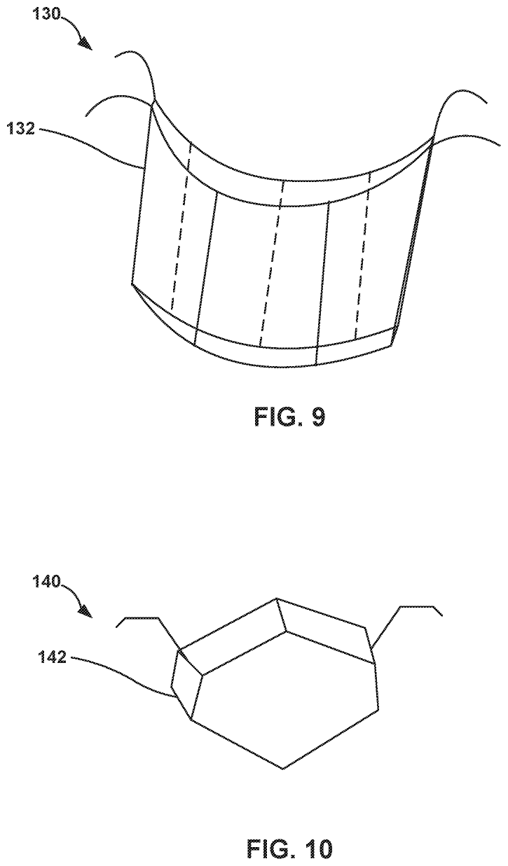

In some examples, rather than a frame of a coaptation zone curtain having a substantially two-dimensional (e.g., planar) shape, the frame may have a three-dimensional shape. FIGS. 9 and 10 are schematic views of example coaptation zone curtains including a three-dimensional frame and curtain. In each of FIGS. 9 and 10, a length of the frame, a depth of the frame, a width of the frame, a curvature of the frame, and a placement and shape of the curtain are selected so that the curtain substantially fills a gap between the at least two valve leaflets and provides a sealing surface between the at least two valve leaflets. For example, three-dimensional frame 132 of coaptation zone curtain 130 shown in FIG. 9 is curved in three-dimensions and the curtain forms an enclosed pouch shape against which the leaflets LF can contact when assuming a coapted position. Similarly, three-dimensional frame 142 of coaptation zone curtain 140 shown in FIG. 10 includes a three-dimensional polygonal shape to which a curtain may be attached to form an enclosed pouch shape against which the leaflets can contact.

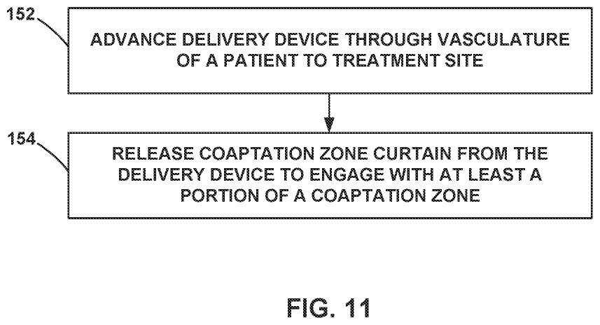

FIG. 11 is a flow diagram illustrating an example technique for implanting a coaptation zone curtain. The technique of FIG. 11 will be described with concurrent reference to coaptation zone curtain 30 of FIG. 3, although it will be understood that the technique of FIG. 11 may be used to implant any of the coaptation zone curtains described herein, and the coaptation zone curtains described herein may be implanted using other techniques.

A delivery device (e.g., delivery device 110 of FIGS. 8A and 8B) may be advanced through vasculature of a patient to a treatment site (152). For example, a clinician may introduce delivery device 110 into vasculature of a patient transcutaneously. For instance, delivery device 110 may be introduced to a femoral or radial artery. Delivery device 110 may be advanced through vasculature of the patient to the treatment site by a clinician manipulating a handle of delivery device 110. In some examples, delivery device 110 may include a steerable shaft or tip to allow the clinician to direct delivery device 110 through bends, curves, and branching points of the vasculature.

In some examples, the treatment site may include the mitral valve, and delivery device 110 may be advanced to the left atrium. In other examples, the treatment site may include another heart valve. Delivery device 110 may access the left atrium trans-septally, trans-aortically, or trans-apically. In some examples, delivery device 110 may be tracked over a guide wire, through a guide catheter, or the like as delivery device 110 is advanced to the treatment site. Delivery device 110 may include one or more radiological markers at or near a distal end of delivery device 110 to assist the clinician in visualizing delivery device 110 as delivery device 110 is advanced to the treatment site.

Once delivery device 110 (e.g., a distal portion of delivery device 110) has been advance to the treatment site, delivery device 110 may release coaptation zone curtain 30, including frame 32 and curtain 34 (154). For example, a clinician may manipulate delivery device 110 to release coaptation zone curtain 30 from a lumen of delivery device 110 to engage with at least a portion of a coaptation zone (e.g., coaptation zone 20 of FIG. 2). The clinician may manipulate delivery device 110 in any of the manners described herein. Delivery device 110 may include a single delivery device having a lumen housing coaptation zone curtain 30 and at least one anchor, a single delivery device having a first lumen housing coaptation zone curtain 30 and a second lumen housing the at least one anchor, or a first delivery device for coaptation zone curtain 30 and a second delivery device for the at least one anchor.

In some examples, delivery device 110 may include a balloon catheter, as described above with respect to FIGS. 8A and 8B. A clinician may inflate the balloon to help deploy coaptation zone curtain 30 in place within the heart 10.

The following clause illustrate example subject matter described herein.

Clause 1. A medical device comprising: a frame configured to substantially conform to a curvature of a coaptation zone of at least two valve leaflets of a cardiac or vascular valve; and a curtain configured to be attached to the frame and extend into the coaptation zone to support coaptation of the at least two valve leaflets.

Clause 2. The medical device of clause 1, wherein the frame comprises a biocompatible metal or alloy.

Clause 3. The medical device of clause 1 or 2, wherein the frame comprises a biocompatible shape memory alloy.

Clause 4. The medical device of clause 3, wherein the frame comprises a nickel-titanium alloy.

Clause 5. The medical device of any one of clauses 1 to 4, wherein the frame comprises a pre-set shape configured to substantially conform to the curvature of at least a portion of the coaptation zone.

Clause 6. The medical device of any one of clauses 1 to 5, wherein the frame defines a substantially two-dimensional shape.

Clause 7. The medical device of any one of clauses 1 to 5, wherein the frame defines a three-dimensional shape.

Clause 8. The medical device of clause 7, wherein at least one of a length of the frame, a depth of the frame, a width of the frame, a curvature of the frame, or a placement of the curtain are selected so that the curtain substantially fills a gap between the at least two valve leaflets and provides a sealing surface between the at least two valve leaflets.

Clause 9. The medical device of any one of clauses 1 to 8, wherein the frame further comprises at least one barb configured to engage tissue of the cardiac or vascular valve.

Clause 10. The medical device of any one of clauses 1 to 9, wherein the frame further comprises at least one alignment marker configured to facilitate alignment of the frame with the coaptation zone.

Clause 11. The medical device of any one of clauses 1 to 10, further comprising at least one anchor configured to anchor the frame to tissue of the cardiac or vascular valve.

Clause 12. The medical device of clause 11, wherein the at least one anchor comprises a helical coil, a conical helical coil, a double helical coil, or a hook.

Clause 13. The medical device of clause 11 or 12, wherein the at least one anchor comprises a biocompatible metal or alloy.

Clause 14. The medical device of any one of clauses 11 to 13, wherein the at least one anchor comprises an attachment feature configured to engage with the frame.

Clause 15. The medical device of any one of clauses 1 to 14, wherein the curtain comprises a biocompatible material.

Clause 16. The medical device of any one of clauses 1 to 15, wherein the curtain comprises porous material.

Clause 17. The medical device of any one of clauses 1 to 15, wherein the curtain comprises substantially non-porous material.

Clause 18. The medical device of any one of clauses 1 to 17, wherein the curtain comprises at least one of pericardial tissue, a biocompatible polymer, or a biocompatible fabric.

Clause 19. The medical device of any one of clauses 1 to 18, wherein the curtain is sewn to the frame.

Clause 20. The medical device of any one of clauses 1 to 19, wherein the cardiac or vascular valve comprises a mitral valve comprising a mitral annulus, an anterior valve leaflet, and a posterior valve leaflet.

Clause 21. A system comprising: a delivery device configured to access vasculature of a patient; and the medical device of any one of clauses 1 to 20, wherein the delivery device is configured to deliver the coaptation zone curtain to the coaptation zone of the cardiac or vascular valve so that the curtain extends between the at least two valve leaflets.

Clause 22. The system of clause 21, wherein the delivery device further comprises a balloon catheter comprising an expandable balloon, and wherein the coaptation zone curtain is disposed over the expandable balloon within a lumen of the delivery device.

Clause 23. The system of clause 22, wherein the expandable balloon is configured to deploy the coaptation zone curtain when the expandable balloon is actuated from an unexpanded state to an expanded state.

Clause 24. The system of any one of clauses 21 to 23, wherein the delivery device is configured to engage at least one anchor with the frame and tissue of the cardiac or vascular valve to anchor the coaptation zone curtain in place.

Clause 25. The system of any one of clauses 21 to 24, wherein the delivery device comprises a radiopaque marker adjacent to a distal end of the delivery device.

Clause 26. A method comprising: advancing a delivery device through vasculature of a patient to a vascular or cardiac treatment site, wherein the delivery device comprises a lumen housing a coaptation zone curtain, wherein the coaptation zone curtain comprises the medical device of any one of clauses 1 to 20; and releasing the coaptation zone curtain from the lumen to the coaptation zone of the cardiac or vascular valve so that the curtain extends between the at least two valve leaflets.

Clause 27. The method of clause 26, wherein the frame comprises a pre-set shape configured to substantially conform to the curvature of a coaptation zone, wherein the frame is in a deformed state within the lumen of the delivery device, and wherein the frame recovers toward the pre-set shape upon being released from the lumen.

Clause 28. The method of clause 26 or clause 27, wherein the frame further comprises at least one barb configured to engage tissue of the cardiac or vascular valve, and wherein releasing the coaptation zone curtain from the lumen comprises engaging the at least one barb with the tissue of the cardiac or vascular valve.

Clause 29. The method of any one of clauses 26 to 28, wherein the frame further comprises at least one alignment marker configured to facilitate alignment of the frame with the coaptation zone, and wherein releasing the coaptation zone curtain from the lumen comprises substantially aligning the frame with the coaptation zone.

Clause 30. The method of any one of clauses 26 to 29, further comprising at least one anchor configured to anchor the frame to tissue of the cardiac or vascular valve, and wherein releasing the coaptation zone curtain from the lumen comprises engaging the at least one anchor with the frame and the tissue of the cardiac or vascular valve.

Clause 31. The method of clause 30, wherein the at least one anchor comprises a helical coil, a conical helical coil, a double helical coil, or a hook.

Clause 32. The method of any one of clauses 30 or 31, wherein the at least one anchor comprises an attachment feature configured to engage with the frame, and wherein engaging the at least one anchor with the frame and the tissue of the cardiac or vascular valve comprises engaging the attachment feature with the frame.

Clause 33. The method of any one of clauses 26 to 32, further comprising attaching the curtain to the frame.

Clause 34. The method of clause 33, wherein attaching the curtain to the frame comprises sewing the curtain to the frame.

Clause 35. The method of clause 33, wherein attaching the curtain to the frame comprises at least one of adhering, clipping, or welding the curtain to the frame.

Clause 36. The method of any one of clauses 26 to 35, wherein the cardiac or vascular valve comprises a mitral valve comprising a mitral annulus, an anterior valve leaflet, and a posterior valve leaflet.

Clause 37. The method of any one of clauses 26 to 36, wherein releasing the coaptation zone curtain from the lumen comprises advancing a balloon catheter comprising an expandable balloon from the lumen, wherein the coaptation zone curtain is disposed over the expandable balloon within the lumen of the delivery device.

Clause 38. The method of clause 37, wherein releasing the coaptation zone curtain from the lumen further comprises expanding the expandable balloon to deploy the coaptation zone curtain.

Various examples have been described. These and other examples are within the scope of the following claims.

What is claimed is:

1. A medical device comprising:
a frame configured to substantially conform to a curvature of a coaptation zone of at least two valve leaflets of a cardiac or vascular valve; and
a curtain configured to be attached to the frame and extend into the coaptation zone to support coaptation of the at least two valve leaflets.

2. The medical device of claim 1, wherein the frame comprises at least one of a biocompatible metal or alloy, biocompatible shape memory alloy, or a nickel-titanium alloy.

3. The medical device of claim 1, wherein the frame comprises a pre-set shape configured to substantially conform to the curvature of at least a portion of the coaptation zone.

4. The medical device of claim 1, wherein at least one of a length of the frame, a depth of the frame, a width of the frame, a curvature of the frame, or a placement of the curtain are selected so that the curtain substantially fills a gap between the at least two valve leaflets and provides a sealing surface between the at least two valve leaflets.

5. The medical device of claim 1, wherein the frame further comprises at least one barb configured to engage tissue of the cardiac or vascular valve.

6. The medical device of claim 1, wherein the frame further comprises at least one alignment marker configured to facilitate alignment of the frame with the coaptation zone.

7. The medical device of claim 1, further comprising at least one anchor configured to anchor the frame to tissue of the cardiac or vascular valve.

8. The medical device of claim 7, wherein the at least one anchor comprises an attachment feature configured to engage with the frame.

9. The medical device of claim 1, wherein the curtain comprises a biocompatible material.

10. The medical device of claim 1, wherein the curtain comprises porous material.

11. The medical device of claim 1, wherein the curtain comprises substantially non-porous material.

12. The medical device of claim 1, wherein the curtain comprises at least one of pericardial tissue, a biocompatible polymer, or a biocompatible fabric.

13. The medical device of claim 1, wherein the curtain is sewn to the frame.

14. The medical device of claim 1, wherein the cardiac or vascular valve comprises a mitral valve comprising a mitral annulus, an anterior valve leaflet, and a posterior valve leaflet.

15. A system comprising:
a delivery device configured to access vasculature of a patient; and
the medical device comprising:
a frame configured to substantially conform to a curvature of a coaptation zone of at least two valve leaflets of a cardiac or vascular valve; and
a curtain configured to be attached to the frame and extend into the coaptation zone to support coaptation of the at least two valve leaflets,
wherein the delivery device is configured to deliver the coaptation zone curtain to the coaptation zone of the cardiac or vascular valve so that the curtain extends between the at least two valve leaflets.

16. The system of claim 15, wherein the delivery device further comprises a balloon catheter comprising an expandable balloon, and wherein the coaptation zone curtain is disposed over the expandable balloon within a lumen of the delivery device.

17. The system of claim 16, wherein the expandable balloon is configured to deploy the coaptation zone curtain when the expandable balloon is actuated from an unexpanded state to an expanded state.

18. The system of claim 15, wherein the delivery device is configured to engage at least one anchor with the frame and tissue of the cardiac or vascular valve to anchor the coaptation zone curtain in place.

19. The system of claim 15, wherein the delivery device comprises a radiopaque marker adjacent to a distal end of the delivery device.

20. A method comprising:
advancing a delivery device through vasculature of a patient to a vascular or cardiac treatment site, wherein the delivery device comprises a lumen housing a coaptation zone curtain, wherein the coaptation zone curtain comprises:
a frame configured to substantially conform to a curvature of a coaptation zone of at least two valve leaflets of a cardiac or vascular valve; and
a curtain configured to be attached to the frame and extend into the coaptation zone to support coaptation of the at least two valve leaflets; and
releasing the coaptation zone curtain from the lumen to the coaptation zone of the cardiac or vascular valve so that the curtain extends between the at least two valve leaflets.

21. The method of claim 20, wherein the frame comprises a pre-set shape configured to substantially conform to the curvature of a coaptation zone, wherein the frame is in a deformed state within the lumen of the delivery device, and wherein the frame recovers toward the pre-set shape upon being released from the lumen.

22. The method of claim 20, wherein the frame further comprises at least one barb configured to engage tissue of the cardiac or vascular valve, and wherein releasing the coaptation zone curtain from the lumen comprises engaging the at least one barb with the tissue of the cardiac or vascular valve.

23. The method of claim 20, wherein the frame further comprises at least one alignment marker configured to facilitate alignment of the frame with the coaptation zone, and wherein releasing the coaptation zone curtain from the lumen comprises substantially aligning the frame with the coaptation zone.

24. The method of claim 20, further comprising at least one anchor configured to anchor the frame to tissue of the cardiac or vascular valve, and wherein releasing the coaptation zone curtain from the lumen comprises engaging the at least one anchor with the frame and the tissue of the cardiac or vascular valve.

25. The method of claim 20, wherein releasing the coaptation zone curtain from the lumen comprises:
advancing a balloon catheter comprising an expandable balloon from the lumen, wherein the coaptation zone curtain is disposed over the expandable balloon within the lumen of the delivery device; and
expanding the expandable balloon to deploy the coaptation zone curtain.
